(19) United States
(12) Patent Application Publication
(10) Pub. No.: US 2020/0315803 A1
(43) Pub. Date: Oct. 8, 2020

(54) SYSTEM AND METHOD FOR FORMING MATERIAL LAYERS FOR SURGICAL APPLICATIONS

(71) Applicant: OsteoMed LLC, Addison, TX (US)

(72) Inventor: Devid R. Zille, Addison, TX (US)

(21) Appl. No.: 16/378,446

(22) Filed: Apr. 8, 2019

(51) Int. Cl. A61F 2/28; A61B 34/10

(52) U.S. Cl. CPC A61F 2/2875; A61B 34/10; A61F 2/30942; A61B 2034/108; A61F 2/2803

(57) ABSTRACT

The present disclosure sets forth a system and method for forming sheets of material, such as titanium mesh or plates, for surgical applications prior to surgery. The disclosed solutions provide this capability without incurring expense from use of PEEK or PEKK by manufacturing contoured plates based on a shape of an anatomical structure in a 3D image, such as a pre-defect MRI. The contoured plates are used to stamp the titanium mesh, plate, or other sheet of material into the shape of the bone prior to the defect. In some aspects, the mesh or other material layer can also be trimmed prior to surgery using, for example, a reproduction of the anatomical structure manufactured from a post-defect MRI of the same anatomical structure.

### TECHNICAL FIELD

The present disclosure generally relates to medical devices, and relates more particularly to a system and method for forming material layers for surgical applications.

### BACKGROUND

Various surgical applications use material layers, such as titanium mesh, titanium plates, or other sheets of material that must be shaped to fit an anatomical structure, such as a bone. Such surgical applications include cranioplasties and surgeries to repair or modify zygomatic, maxillary, and mandibular anatomical structures. Further surgical applications include surgeries that use meshes applied to long bones, and surgeries that use bent plates for extremities, such as the hands and feet. The present disclosure primarily presents examples with respect to cranioplasty, but the principles, techniques, and devices described herein are also applicable to these other surgical applications.

Cranioplasty is the surgical intervention to repair cranial defects in both cosmetic and functional ways. Cranial defects usually occur after trauma, neurosurgical procedures like decompressive craniotomy, tumor resections, infection and congenital defects. The purpose of cranial vault repair is to protect the underlying brain tissue, to reduce any localized pain and patient anxiety, and improve cranial aesthetics. Cranioplasty is a frequent neurosurgical procedure achieved with the aid of cranial prostheses made from materials such as: titanium, autologous bone, ceramics and polymers. Implant customized manufacturing for cranioplasties allows for a precise and anatomical reconstruction in a shorter operating time compared to other conventional techniques. However, prosthesis production is often costly and can require complex intraoperative processes.

Many different types of materials have been used throughout the history of cranioplasty. Cranioplasty was first documented by Fallopius who described repair using gold plates; the first bone graft was documented by van Meekeren. The first significant improvement for this procedure began with experimentation involving bone grafts in the late 19th century as a more natural approach for repairing cranial defects. The next impetus for advancement came because of wartime injuries incurred during World Wars I and II and involved experimentation with synthetic materials to counter the common complications associated with bone grafts. Methyl methacrylate, hydroxyapatite, ceramics, and poly ether ether ketone implants among other materials have since been researched and used. Materials currently of most interest include polymethyl methacrylate (PMMA), titanium, and poly-ether-ether-ketone (PEEK).

Polymethyl methacrylate (PMMA), is commonly known as bone cement, and is widely used for implant fixation in various orthopaedic and trauma surgery. In reality, “cement” is a misnomer because, the word cement is used to describe a substance that bonds two things together. However, PMMA acts as a space-filler that creates a tight space which holds the implant against the bone and thus acts as a ‘grout’. Bone cements have no intrinsic adhesive properties, but they rely instead on close mechanical interlock between the irregular bone surface and the prosthesis.

PEEK is the preferred material for cranioplasty implants in hospitals around the world. In many countries, it is the “gold standard” for large defects and also well suited for small and standard defects. Benefits of PEEK include toughness, durability, similarity to cortical bone, receptivity to common titanium fixation and screws, bio-inertness, radiolucency, customizable fit and ability to provide finely tapered edges, easy removal if needed, and patient-specific manufacture for minimal modification in the operating room. However, PEEK is notoriously expensive and also may not support soft tissue adhesion due to its inert characteristics.

PEKK (poly ether ketone ketone) is a 3D-printed variant similar to PEEK material, operating in much the same way as PEEK. The 3D-printing process of PEKK leaves pores in the implant that the manufacturer sells as “promoting bone growth” while some would flag the implant as a sterilization risk. Benefits of PEKK include minor osseointegration, patient-specific manufacture, and similarity to cortical bone. However, the porous characteristics of PEKK yield a potential sterilization risk, and its inert characteristics produce a potential lack of soft tissue adhesion.

The flexible structure of steel and deformities in the material seen after minor traumas prevented its use in large defects. In contrast, titanium is difficult to shape, but relatively cheaper, biocompatible, and radiolucent after mixing with other metals. It also exhibits good resistance to infection, even when in contact with the paranasal sinuses. Recently, titanium meshes have been used as a support to cement materials. In this way, the strong resistance against mechanical stress of the titanium and the ability to remodel the cement materials were combined. For these reasons, some consider titanium mesh and/or plates to be a better solution for cranial reconstruction. However, surgeons are required to shape the titanium mesh in the operating room during surgery and trim the edges of the mesh. This procedure is difficult and time consuming, which can cause complications for a patient.

### SUMMARY

The present disclosure sets forth a system and method for pre-forming sheets of material, such as titanium mesh, for bone reconstruction prior to surgery. The disclosed solutions provide this capability without incurring expense from use of PEEK or PEKK. In some aspects, the mesh can also be trimmed prior to surgery. These and other benefits will become apparent from the disclosure set forth herein.

In an aspect, a method of manufacturing a stamping apparatus includes accessing a computer-readable medium having stored thereon a three-dimensional (3D) image of an anatomical structure. The method additionally includes 3D printing a first contoured plate based on a contoured surface of the anatomical structure of the 3D image. The method also includes 3D printing a second contoured plate based on the contoured surface of the anatomical structure of the 3D image. The method further includes providing one or more guide members to the first contoured plate and the second contoured plate. The one or more guide members are configured to maintain an aligned positional relationship of the first contoured plate and the second contoured plate during a stamping operation that employs the first contoured plate and the second contoured plate to stamp a sheet of material into a shape that substantially matches a shape of the anatomical structure.

In another aspect, a stamping apparatus has a first contoured plate manufactured based on a contoured surface of an anatomical structure of a 3D image, and a second contoured plate manufactured based on the contoured surface of the anatomical structure of the 3D image. The apparatus also has one or more guide members provided to the first contoured plate and the second contoured plate. The one or more guide members are configured to maintain an aligned positional relationship of the first contoured plate and the second contoured plate during a stamping operation that employs the first contoured plate and the second contoured plate to stamp a sheet of material into a shape that substantially matches a shape of the anatomical structure.

In another aspect, a method of manufacturing a medical device includes positioning a sheet of material between a first contoured plate and a second contoured plate, the first contoured plate and the second contoured plate being manufactured based on a contoured surface of an anatomical structure of a three-dimensional (3D) image. The first contoured plate and the second contoured plate are provided with one or more guide members configured to maintain an aligned positional relationship of the first contoured plate and the second contoured plate during a stamping operation that employs the first contoured plate and the second contoured plate to stamp the sheet of material into a shape that substantially matches a shape of the anatomical structure. The method additionally includes moving at least one of the first contoured plate or the second contoured plate to perform the stamping operation.

In another aspect, a medical apparatus has a first contoured plate three-dimensionally (3D) printed based on a contoured surface of an anatomical structure of a 3D image, and a second contoured plate 3D printed based on the contoured surface of the anatomical structure of the 3D image. The apparatus additionally has one or more guide members provided to the first contoured plate and the second contoured plate. The one or more guide members are configured to maintain an aligned positional relationship of the first contoured plate and the second contoured plate during a stamping operation that employs the first contoured plate and the second contoured plate to stamp a sheet of material into a shape that substantially matches a shape of the anatomical structure. The apparatus also has the sheet of material having the shape and positioned between the first contoured plate and the second contoured plate.

In another aspect, an apparatus has a plate pre-formed to fit a shape of an anatomical structure. The plate is pre-formed by accessing a computer-readable medium having stored thereon a three-dimensional (3D) image of an anatomical structure, and 3D printing the plate based on a contoured surface of the anatomical structure of the 3D image. The plate is also pre-formed by providing one or more guide members to the plate. The one or more guide members are configured to maintain an aligned positional relationship of the plate and another plate during a stamping operation that employs the plate and the other plate to stamp a sheet of material into a shape that substantially matches a shape of the anatomical structure.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding, reference is now made to the following descriptions taken in conjunction with the accompanying drawings, in which:

FIG. 1 is a block diagram illustrating example blocks of a method of manufacturing a stamping apparatus in accordance with embodiments of the present disclosure;

FIG. 2 is a block diagram illustrating an exemplary system for manufacturing a stamping apparatus in accordance with embodiments of the present disclosure;

FIG. 3 is an isometric view featuring a top of a lid and sides of a receptacle of a stamping apparatus in accordance with embodiments of the present disclosure;

FIG. 4 is an isometric view featuring a bottom and sides of a receptacle of a stamping apparatus in accordance with embodiments of the present disclosure;

FIG. 5 is a block diagram illustrating example blocks of a method of manufacturing a medical device in accordance with embodiments of the present disclosure;

FIG. 6 is an isometric view featuring a medical device positioned between contoured plates of a stamping apparatus in accordance with embodiments of the present disclosure;

FIG. 7 is an isometric view featuring movement of the plates of the stamping apparatus during a stamping operation in accordance with embodiments of the present disclosure;

FIG. 8 is an isometric view of the plates separated to show a stamped sheet of material having a shape resulting from the stamping operation in accordance with embodiments of the present disclosure;

FIG. 9 is a block diagram illustrating example blocks of another method of manufacturing a medical device in accordance with embodiments of the present disclosure;

FIG. 10 is an isometric view of a stamped sheet of material aligned with a reproduction of an anatomical structure for trimming in accordance with embodiments of the present disclosure;

FIG. 11 is an isometric view of a stamped sheet of material surgically implanted by attachment to an anatomical structure in accordance with embodiments of the present disclosure.

FIG. 12 is an isometric view of stamped sheets of material attached to a reproduction of an anatomical structure in accordance with embodiments of the present disclosure.

FIG. 13 is an isometric view of stamped sheets of material attached to a reproduction of an anatomical structure in accordance with embodiments of the present disclosure.

### DETAILED DESCRIPTION

The detailed description set forth below, in connection with the appended drawings, is intended as a description of various possible configurations and is not intended to limit the scope of the disclosure. Rather, the detailed description includes specific details for the purpose of providing a thorough understanding of the inventive subject matter. It will be apparent to those skilled in the art that these specific details are not required in every case and that, in some instances, well-known structures and components are shown in block diagram form for clarity of presentation.

It is appreciated that much of the discussion herein utilizes a skull structure and repairs defects to the same. This discussion is provided by way of example and embodiments of the present application are not limited to this particular use. A person of ordinary skill in the art would understand that the systems and methods disclosed herein are applicable to other areas of the body where shaped objects may be utilized for forming/reconstruction of a surface area.

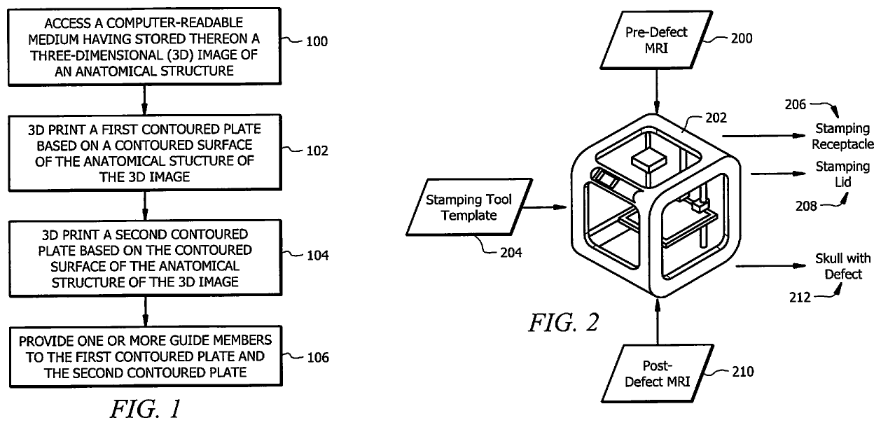

FIG. 1 is a block diagram illustrating example blocks of a method of manufacturing a stamping apparatus in accordance with embodiments of the present disclosure. Beginning at block 100, the method includes accessing a computer-readable medium having stored thereon a three-dimensional (3D) image of an anatomical structure. For example, block 100 may include accessing a computer memory or drive having an MRI of a body part, such as a human head, with respect to which the anatomical structure may be a human skull. The 3D image may be a processed image that interpolates an appearance and contour of a desired (e.g., damage and/or defect-free) structure. For example, a contour to correct damage to a skull on one side may be interpollated by digitally inverting an image of the opposite side of the skull. As another example, a reproduction of a damaged anatomical structure may be 3D printed from an MRI of the damged or defective structure, and the reproduction may be repaired with cement or another filler. The resulting reproduction of a repaired anatomical structure may then be imaged. For plastic surgery applications, an ideal contour may be created in a smilar manner by removing bone and/or adding filler, as desired. Source images may also include images taken from earlier scans of the patient (e.g., an MRI of the patient's head from before an injury), or from a repository of images where an image may be selected that most closely approximates the desired end-resulting structure. In sum there at least three techniques for creating an image to reconstruct a defect: (1) mirroring the contralateral side in cases of unilateral defects; (2) using an exam from a repository; and/or (3) free-hand modelling using the patient's defect boundaries as reference. Processing may proceed from block 100 to block 102.

At block 102, the method continues by 3D printing a first contoured plate based on a contoured surface of the anatomical structure of the 3D image (e.g., a positive image). For example, block 102 may include using a 3D printer or a subtractive 3D printer, such as a CNC machine, to manufacture a plate based on part of the human skull in the MRI accessed in block 100. In some implementations, this part of the human skull may correspond to a portion that exhibits a post craniotomy defect. The part of the human skull may be integrated with another structure, such as a plate surface, features for attachment to a pressing surface of a linear or angular press, a stackable tray, a receptacle, etc. Accordingly, the manufactured plate may correspond to a plate, tray, receptacle, etc., having a portion thereof that exhibits a contour that matches a contour of a surface of the anatomical structure, such as the portion of the human skull. Processing may proceed from block 102 to block 104.

At block 104, the method continues by 3D printing a second contoured plate based on the contoured surface of the anatomical structure of the 3D image (e.g., a negative image). For example, block 104 may include using the 3D printer or the subtractive 3D printer, such as the CNC machine, to manufacture the plate based on the part of the human skull in the MRI accessed in block 100 and used at block 102. The part of the human skull may also be integrated with another structure, such as a plate surface, a plate surface having features for attachment to a pressing surface of a linear or angular press, a stackable tray, a receptacle lid, etc. Accordingly, the manufactured plate may correspond to a plate, tray, receptacle lid, etc., having a portion thereof that exhibits the same contour as that of the first plate. Processing may proceed from block 104 to block 106.

At block 106, the method includes providing one or more guide members to the first contoured plate and the second contoured plate. The one or more guide members are configured to maintain an aligned positional relationship of the first contoured plate and the second contoured plate during a stamping operation that employs the first contoured plate and the second contoured plate to stamp a sheet of material into a shape that substantially matches a shape of the anatomical structure. For example, the plates may be attached to opposing pressure surfaces of a linear or angular press. Alternatively, integrating plates with other structures that correspond to stackable trays provides tray edges that serve as the guide members to maintain the aligned position of the plates as the contoured surfaces are brought together during a stamping operation. Similarly, integrating the contoured surfaces with a receptacle and lid having sides that serve as the guide members to maintain the aligned position of the plates as the contoured surfaces are brought together during a stamping operation. In this case, a first dimension of the sides of the first contoured plate is smaller than a second dimension of the one or more circumferential sides of the second contoured plate, and the first dimension and second dimension are sized to achieve a sliding fit of the one or more sides of the first contoured plate within the one or more sides of the second contoured plate.

At blocks 102 and 104, a benefit of reduced cost may be achieved if the first contoured plate and the second contoured plate are composed primarily of a plastic material. It should be understood that 3D printers may produce plates of plastic, resin, and/or metal material, and that the plates may be printed separately or at a same time, with a gap between the plates. Also, subtractive 3D printers may produce shapes in plastic, foam, etc. The plates may be composed of the same or different materials, or a mixture of materials. It is envisioned that the sheet of material stamped by the plates corresponds to a titanium mesh or a titanium plate.

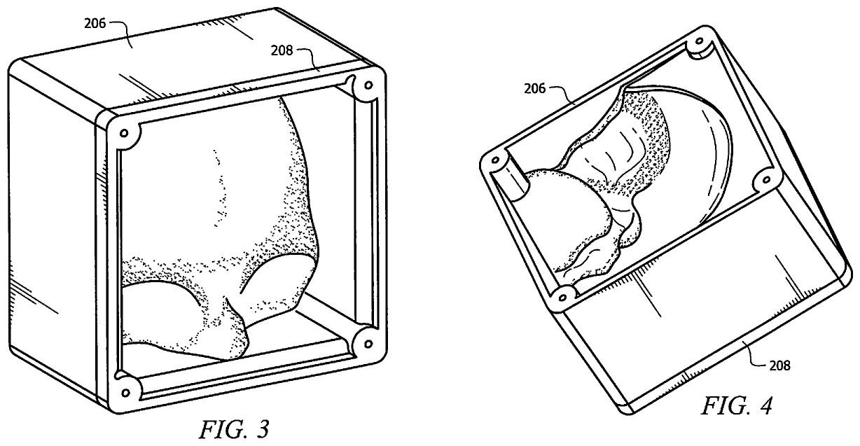

FIG. 2 is a block diagram illustrating an exemplary system for manufacturing a stamping apparatus in accordance with embodiments of the present disclosure. A 3D image, such as a pre-defect MRI or a processed image, may be provided to a 3D printer 202 having a stamping tool template 204 that, in this example, corresponds to a receptacle and lid. The 3D printer prints the stamping receptacle 206 and the stamping lid 208 with a contour provided to each that is based on a designated portion of a surface of a bone of the pre-defect MRI 200. In some implementations, a post-defect MRI 210 is also provided to the 3D printer 202, and the 3D printer also prints a reproduction of a bone having a defect, such as a portion of a skull having a portion of bone removed during a craniotomy. The defect may be caused by removal of a bone flap in a region of the designated portion of the surface of the bone of the pre-defect MRI 200. This reproduction of the bone exhibiting the defect may be used to guide trimming of a medical device created using the plates. The reproduction allows the trimming to be performed before, rather than during, the cranioplasty, and attachment points may also be determined prior to surgery, thus saving time in the operating room.

FIG. 3 is an isometric view featuring a top of a lid 208 and sides of a receptacle 206 of a stamping apparatus in a closed/stamped configuration in accordance with embodiments of the present disclosure. The sides shown are generally square so as to form a cube shape, but it is envisioned that the lid 208 and receptacle 206 may have curved (circular, oval, etc.) sides such that the lid 208 and receptacle 206 each have only one side. The lid 208 has a rim that rests upon the sides of the receptacle 206, and sides that extend down into the receptacle 206. Accordingly, a dimension (e.g., length, width, radius) of at least part of the sides of the lid may be smaller than another dimension (e.g., length, width, radius) of the sides of the receptacle 206, thus achieving a sliding fit of the lid 208 within the receptacle 206. The contour formed in the lid remains aligned with the same contour formed in the receptacle during a stamping operation.

FIG. 4 is an isometric view featuring a bottom and sides of the receptacle 206 of the stamping apparatus in accordance with embodiments of the present disclosure. The contour formed in the bottom of the receptacle 206 is aligned with the same contour formed in the lid 208. Threaded through holes for receiving screws or bolts may be provided to the receptacle 206, and corresponding through holes in the lid 208 permit threaded members to be inserted and actuated to apply pressure to a sheet of material sandwiched between the contoured surfaces of the receptacle 206 and lid 208. The threaded members (e.g., screws, bolts, etc.) may be left in place during storage and/or transport to protect the shaped sheet of material and, with an air tight fit, keep it sterile until time of use.

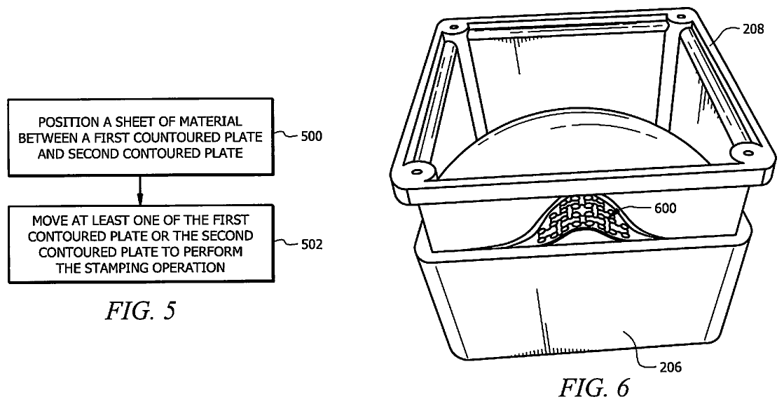

FIG. 5 is a block diagram illustrating example blocks of a method of manufacturing a medical device in accordance with embodiments of the present disclosure. Beginning at block 500, the method includes positioning a sheet of material between a first contoured plate and a second contoured plate, the first contoured plate and the second contoured plate being manufactured based on a contoured surface of an anatomical structure of a three-dimensional (3D) image. The first contoured plate and the second contoured plate are also provided with one or more guide members configured to maintain an aligned positional relationship of the first contoured plate and the second contoured plate during a stamping operation that employs the first contoured plate and the second contoured plate to stamp the sheet of material into a shape that substantially matches a shape of the anatomical structure. The method may proceed from block 500 to block 502.

At block 502, the method continues by moving at least one of the first contoured plate or the second contoured plate to perform the stamping operation. For example, in the case of a receptacle and lid, the lid may be moved into the receptacle with sufficient pressure to impart the shape to the sheet of material sandwiched between the contoured surfaces of the lid and receptacle. As noted above, mechanical devices, such as screws or bolts, may be used to apply the pressure, but in many embodiments hand-force is sufficient to stamp the plate. A similar technique may be used with stackable trays, as discussed above. Alternatively, the contoured plates may be implemented in a linear or angular press, and the movement may be effected by actuating the press. The plates may be separated and/or joined together by any suitable means (e.g., screws, bolts, tape, clasps, binding, etc.) in order to secure the stamped sheet of material between the plates for storage and/or transportation.

The contoured plates and the medical device combine to form a kit of parts for transportation. In the case of a receptacle and lid, the guide members aid in maintaining alignment of the plates while the medical device is stored therein. Additional medical devices may also be sent as part of the kit as extras to be used by the surgeon if needed. In such example embodiments, the surgeon may utilize the stamping mechanism to form an additional plate in the event that the original plate is damaged. As discussed above, a reproduction of the defective anatomical structure may be created to aid in trimming of the medical device and determination of attachment points by the surgeon prior to the cranioplasty. This reproduction may also be included as part of the kit.

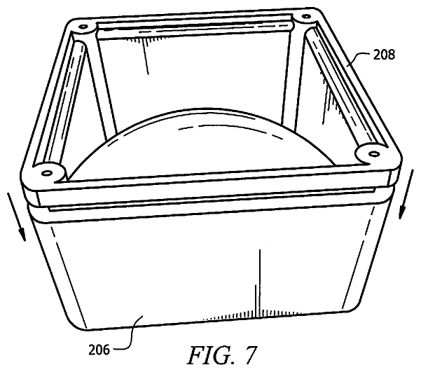

FIG. 6 is an isometric view featuring a medical device positioned between contoured plates of a stamping apparatus in accordance with embodiments of the present disclosure. A sheet of material 600, such as titanium mesh, is positioned between the plates, which in this example are depicted as a lid 208 and receptacle 206. The sides of the lid 208 are dimensioned to achieve a sliding fit within the sides of receptacle 208 as the lid 208 is pressed into the receptacle 206.

FIG. 7 is an isometric view featuring movement of the plates of the stamping apparatus during a stamping operation in accordance with embodiments of the present disclosure. As shown, the lid 208 has been moved by pressing the lid 208 down into the receptacle 206. The matching contours of the lid 208 and receptacle 206 are brought together in the stamping operation with sufficient pressure to stamp the sheet of material into the shape of the anatomical structure.

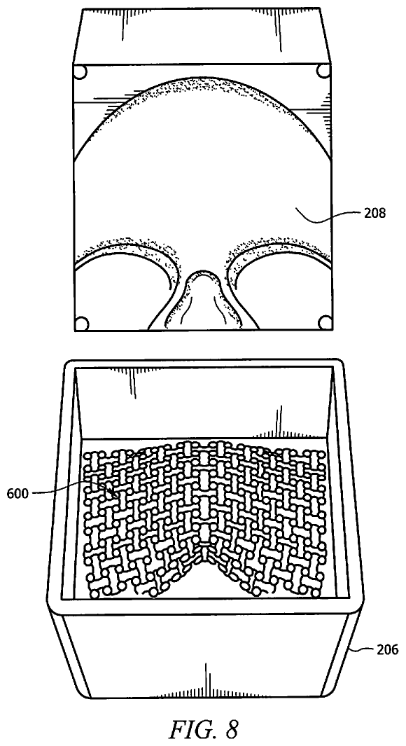

FIG. 8 is an isometric view of the plates separated to show a stamped sheet of material 600 having a shape resulting from the stamping operation in accordance with embodiments of the present disclosure. In this example, the plates are separated by removing the lid 208 from the receptacle 206. The stamped sheet of material 600 is a medical device produced as a result of the stamping operation.

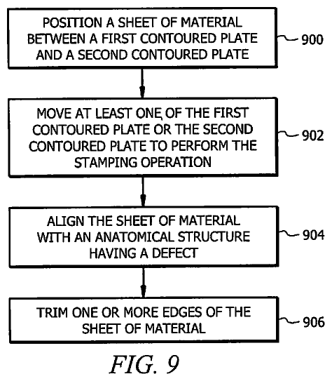

FIG. 9 is a block diagram illustrating example blocks of another method of manufacturing a medical device in accordance with embodiments of the present disclosure.

Beginning at block 900, the method includes positioning a sheet of material between a first contoured plate and a second contoured plate, the first contoured plate and the second contoured plate being manufactured based on a contoured surface of an anatomical structure of a three-dimensional (3D) image. The first contoured plate and the second contoured plate are also provided with one or more guide members configured to maintain an aligned positional relationship of the first contoured plate and the second contoured plate during a stamping operation that employs the first contoured plate and the second contoured plate to stamp the sheet of material into a shape that substantially matches a shape of the anatomical structure. The method may proceed from block 900 to block 902.

At block 902, the method continues by moving at least one of the first contoured plate or the second contoured plate to perform the stamping operation. For example, in the case of a receptacle and lid, the lid may be moved into the receptacle with sufficient pressure to impart the shape to the sheet of material sandwiched between the contoured surfaces of the lid and receptacle. The plates may be separated to permit removal of the resulting medical device. The method may proceed from block 902 to block 904.

At block 904, the method continues by aligning the stamped sheet of material with an anatomical structure having a defect, such as a human skull or a reproduction of a portion of the human skull. For example, a titanium mesh may be placed over the defect and attached to the anatomical structure with screws and/or adhesive, clamped in place, or held in place. The method may proceed from block 904 to block 906.

At block 906, the method continues by trimming one or more edges of the sheet of material. For example, a reproduction of the anatomical structure may have a shape such that edges thereof may guide trimming of the sheet of material, such as a titanium mesh. Any rough, sharp or pointed edges that remain may be ground down and polished to avoid causing irritation upon implantation. Using a reproduction of the anatomical structure to accomplish the trimming prior to surgery advantageously avoids generating metallic shards or powders in the operating room during surgery. The finished medical device may then be secured between the plates for storage and/or transport, as previously described. Sterilization of the plates and/or medical device may optionally be used prior to storage and transport.

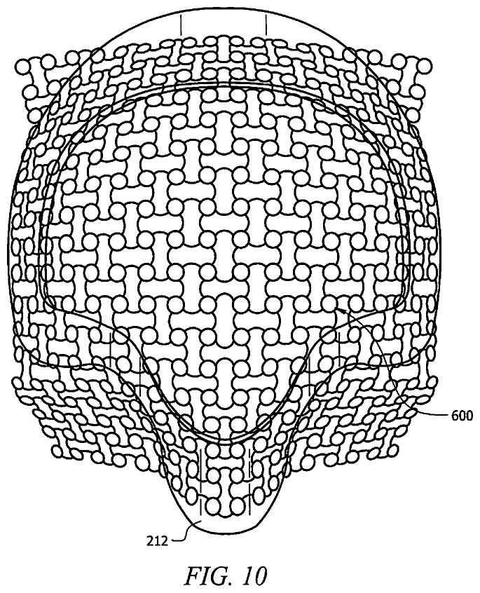

FIG. 10 is an isometric view of a stamped sheet of material 600 aligned with a reproduction of an anatomical structure 212 for trimming in accordance with embodiments of the present disclosure. The sheet of material 600 shown is a titanium mesh that has been stamped into a shape of a pre-defect anatomical structure, as previously described. The reproduction of an anatomical structure 212 produced from a post-craniotomy MRI has a shape that guides trimming of edges of the titanium mesh prior to surgery. Using such a shaped reproduction of the post-defect anatomical structure to accomplish the trimming prior to surgery advantageously allows the surgeon to prepare the medical device ahead of time and determine attachment points, which saves time in the operating room. Additionally, trimming the mesh before, instead of during, the cranioplasty avoids generating metallic shards or powders in the operating room during surgery. The finished medical device may then be secured between the plates for storage and/or transport, as previously described. Sterilization of the plates and/or medical device may optionally be used prior to storage and transport.

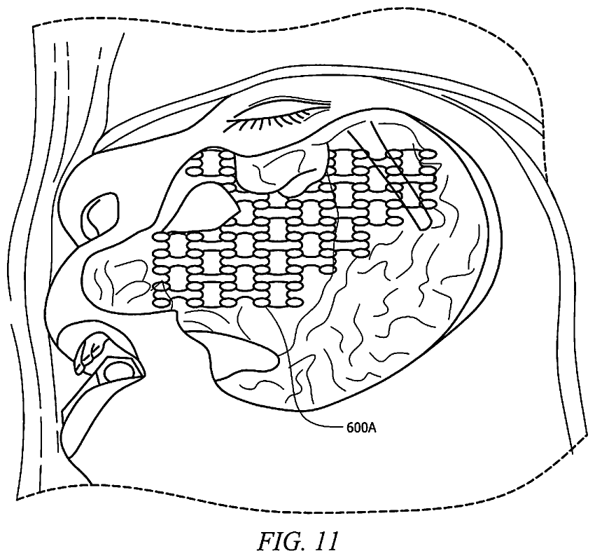

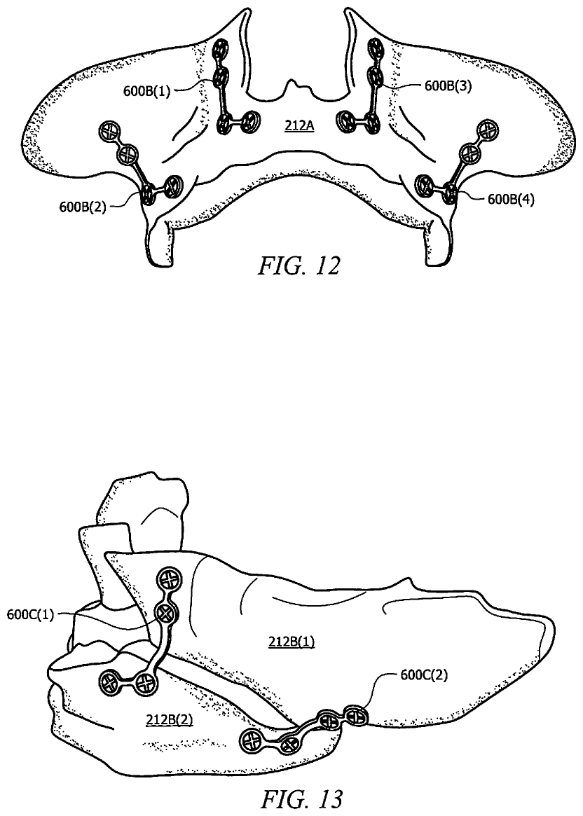

Although the present disclosure describes use of the stamping apparatus to manufacture a medical device suitable for use in cranioplasty, it is envisioned that the systems and methods described herein are not limited to such applications. For example, the 3D image used to generate the contoured plates may be any type of 3D image, allowing for applications in cosmetic surgery. Other applications include, but are not limited to: (1) forming meshes for midface and zygomatic reconstructions; (2) forming meshes for mandibular reconstructions; (3) forming plates (bend) for any type of orthognathic surgery; (4) forming plates (bend) for any type of reconstruction (e.g., Maxilla, Mandible, etc.); (5) forming meshes for long bones; and/or (6) forming plates (bend) for extremity surgeries (e.g., hands or feet). FIG. 11, for example, illustrates a sheet of material 600A that is a titanium mesh shaped into a medical device according to the techniques described herein and implanted for orbital reconstruction. Also, FIG. 12 illustrates sheets of material 600B(1)-600B(4) that are titanium plates bent into medical devices according to the techniques described herein and attached to a reproduction of an anatomical structure 212A corresponding to maxillary bones. Further, FIG. 13 illustrates sheets of material 600C(1) and 600C(2) that are titanium plates bent into medical devices according to the techniques described herein and attached to a reproduction of an anatomical structure 212B(1) and 212B(2) corresponding to mandibular bones. Further applications will be readily apparent to one skilled in the art.

The functional blocks and modules described herein (e.g., the functional blocks and modules in FIGS. 1, 2, 5 and 9) may comprise processors, electronics devices, hardware devices, electronics components, logical circuits, memories, software codes, firmware codes, etc., or any combination thereof.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the disclosure herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. Computer-readable storage media may be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, a connection may be properly termed a computer-readable medium. For example, if the software is transmitted from a website, server, or other remote source using a coaxial cable, fiber optic cable, twisted pair, or digital subscriber line (DSL), then the coaxial cable, fiber optic cable, twisted pair, or DSL, are included in the definition of medium. Disk and disc, as used herein, includes compact disc (CD), laser disc, optical disc, digital versatile disc (DVD), hard disk, solid state disk, and blu-ray disc where disks usually reproduce data magnetically, while discs reproduce data optically with lasers. Combinations of the above should also be included within the scope of computer-readable media.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C) or any of these in any combination thereof.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein but is to be accorded the widest scope consistent with the principles and novel features disclosed herein.

Although embodiments of the present application and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps. Moreover, the scope of the present application is not intended to be limited to the particular embodiments of the process, machine, manufacture, composition of matter, means, methods and steps described in the specification.

1. A method of manufacturing a stamping apparatus, the method comprising:
accessing a computer-readable medium having stored thereon a three-dimensional (3D) image of an anatomical structure;
3D printing a first contoured plate based on a contoured surface of the anatomical structure of the 3D image;
3D printing a second contoured plate based on the contoured surface of the anatomical structure of the 3D image; and
providing one or more guide members to the first contoured plate and the second contoured plate, wherein the one or more guide members are configured to maintain an aligned positional relationship of the first contoured plate and the second contoured plate during a stamping operation that employs the first contoured plate and the second contoured plate to stamp a sheet of material into a shape that substantially matches a shape of the anatomical structure.

2. The method of claim 1, wherein providing the one or more guide members comprises:
3D printing the first contoured plate with one or more sides; and
3D printing the second contoured plate with one or more sides,
wherein a first dimension of the sides of the first contoured plate is smaller than a second dimension of the one or more circumferential sides of the second contoured plate, the first dimension and second dimension being sized to achieve a sliding fit of the one or more sides of the first contoured plate within the one or more sides of the second contoured plate.

3. The method of claim 1, wherein the first contoured plate and the second contoured plate are composed primarily of a plastic material.

4. The method of claim 1, wherein the sheet of material corresponds to at least one of a titanium mesh or a titanium plate.

5. The method of claim 1, wherein the 3D image is a magnetic resonance image (MRI), and the anatomical structure is at least part of a bone.

6. A stamping apparatus, the apparatus comprising:
a first contoured plate defining a contoured surface of an anatomical structure wherein the contoured surface of the anatomical structure corresponds to a desired defect-free anatomical structure for a particular patient having an anatomical structural defect;
a second contoured plate manufactured based on the contoured surface of the anatomical structure;
a sheet of material configured to be inserted between the first contoured plate and second contoured plate; and
wherein the first contoured plate and the second contoured plate are configured to maintain an aligned positional relationship during a stamping operation that employs the first contoured plate and the second contoured plate to stamp the sheet of material into a shape that substantially matches a shape of the desired defect-free anatomical structure.

7. The stamping apparatus of claim 6, further comprising one or more guide members that correspond to one or more sides integrally formed with the first contoured plate and one or more sides integrally formed with the second contoured plate, wherein a first dimension of the sides of the first contoured plate is smaller than a second dimension of the one or more sides of the second contoured plate, the first dimension and second dimension being sized to achieve a sliding fit of the one or more sides of the first contoured plate within the one or more sides of the second contoured plate.

8. The stamping apparatus of claim 6, wherein the first contoured plate and the second contoured plate are composed primarily of a plastic material.

9. The stamping apparatus of claim 6, wherein the sheet of material is at least one of a titanium mesh or a titanium plate.

10. The stamping apparatus of claim 6, wherein the 3D image is an image from a magnetic resonance image (MRI) machine, and the anatomical structure is at least part of a bone.

11. A method of manufacturing a medical device, comprising:
positioning a sheet of material between a first contoured plate and a second contoured plate, the first contoured plate and the second contoured plate being manufactured based on a contoured surface of an anatomical structure of a three-dimensional (3D) image, the first contoured plate and the second contoured plate being provided with one or more guide members configured to maintain an aligned positional relationship of the first contoured plate and the second contoured plate during a stamping operation that employs the first contoured plate and the second contoured plate to stamp the sheet of material into a shape that substantially matches a shape of the anatomical structure; and
moving at least one of the first contoured plate or the second contoured plate to perform the stamping operation.

12. The method of claim 11, wherein the one or more guide members correspond to one or more sides integrally formed with the first contoured plate and one or more sides integrally formed with the second contoured plate, wherein a first dimension of the sides of the first contoured plate is smaller than a second dimension of the one or more circumferential sides of the second contoured plate, the first dimension and second dimension being sized to achieve a sliding fit of the one or more sides of the first contoured plate within the one or more sides of the second contoured plate.

13. The method of claim 11, wherein the first contoured plate and the second contoured plate are composed primarily of a plastic material.

14. The method of claim 11, wherein the sheet of material corresponds to at least one of a titanium mesh or a titanium plate.

15. The method of claim 11, wherein the 3D image is a magnetic resonance image (MRI), and the anatomical structure is at least part of a bone.

16. A apparatus, comprising:
a first contoured plate three-dimensionally (3D) printed based on a 3D image of a contoured surface of an anatomical structure wherein the contoured surface of the anatomical structure corresponds to a desired defect-free anatomical structure for a particular patient having an anatomical structural defect;
a second contoured plate 3D printed based on the 3D image of a contoured surface of the anatomical structure;
a sheet of material configured to be positioned within and between the first contoured plate and the second contoured plate; and
wherein the first and second contoured plates include one or more guide members, the one or more guide members configured to maintain an aligned positional relationship of the first contoured plate and the second contoured plate during a stamping operation that employs the first contoured plate and the second contoured plate to stamp a sheet of material into a shape that substantially matches a shape of the anatomical structure.

17. The medical apparatus of claim 16, wherein the one or more guide members correspond to one or more sides integrally formed with the first contoured plate and one or more sides integrally formed with the second contoured plate, wherein a first dimension of the sides of the first contoured plate is smaller than a second dimension of the one or more circumferential sides of the second contoured plate, the first dimension and second dimension being sized to achieve a sliding fit of the one or more sides of the first contoured plate within the one or more sides of the second contoured plate.

18. The medical apparatus of claim 16, wherein the first contoured plate and the second contoured plate are composed primarily of a plastic material.

19. The medical apparatus of claim 16, wherein the sheet of material is at least one of a titanium mesh or a titanium plate.

20. The medical apparatus of claim 16, wherein the 3D image is an image from a magnetic resonance image (MRI) machine, and the anatomical structure is at least part of a bone.

21. An apparatus, comprising:
a plate pre-formed to fit a shape of an anatomical structure, the plate being pre-formed by:
accessing a computer-readable medium having stored thereon a three-dimensional (3D) image of the anatomical structure wherein the anatomical structure corresponds to a desired defect-free anatomical structure for a particular patient having an anatomical structural defect;
3D printing the plate to include a contoured surface corresponding to the 3D image of the anatomical structure; and
configuring the plate with one or more guide members, wherein the one or more guide members are configured to maintain an aligned positional relationship of the plate and another plate during a stamping operation that employs the plate and the other plate to stamp a sheet of material into a shape that substantially matches the shape of the anatomical structure.

22. The apparatus of claim 21, wherein the one or more guide members correspond to one or more sides integrally formed with the plate, wherein a first dimension of the sides of the plate is at least one of smaller or larger than a second dimension of one or more sides of the other plate, the first dimension and second dimension being sized to achieve a sliding fit of the one or more sides of the first contoured plate and the one or more sides of the second contoured plate.

23. The method of claim 21, wherein the plate is composed primarily of a plastic material.

24. The method of claim 21, wherein the sheet of material is at least one of a titanium mesh or a titanium plate.

25. The method of claim 21, wherein the 3D image is an image from a magnetic resonance image (MRI) machine, and the anatomical structure is at least part of a bone.
